(19) United States
(12) Patent Application Publication
(10) Pub. No.: US 2012/0259295 A1
(43) Pub. Date: Oct. 11, 2012

(54) Medical Product with Biodegradable Portion

(76) Inventor: Peter M. Bonutti, Effingham, IL (US)

(21) Appl. No.: 13/452,825

(22) Filed: Apr. 20, 2012

Related U.S. Application Data
(63) Continuation of application No. 11/123,497, filed on May 5, 2005.
(60) Provisional application No. 60/568,620, filed on May 6, 2004.

(51) Int. Cl. C08G 63/06; A61J 1/10; A61B 17/3211; A61M 5/00

(52) U.S. Cl. 604/272; 528/361; 604/403; 606/167

(57) ABSTRACT

Embodiments of the present disclosure provide a medical product with a biodegradable portion. The biodegradable portion is made of a biodegradable material and the biodegradable portion degrades faster than the remaining portion of the medical product.

### CROSS-REFERENCE TO RELATED APPLICATION

This application is a continuation of U.S. application Ser. No. 11/123,497, filed May 5, 2005, which claims the benefit under 35 U.S. §119(e) of U.S. Provisional Application No. 60/568,620, filed May 6, 2004.

### BACKGROUND

Approximately 60.5 billion pounds of plastic materials were produced in the United States in 1991, of which approximately 15 billion pounds were one-way, or non-returnable, plastics used in packaging. A significant amount of these plastic materials are discarded and become pollutants that deface the landscape and threaten wildlife. At least about one million seabirds and about 100,000 marine mammals die each year as the result of plastic pollutants. Degradable disposable materials have become a replacement for the tremendous amount of conventional plastic materials which, when discarded, do not degrade well.

Disposable materials can degrade in a variety of ways, including, but not limited to, hydrolytic, biological, chemical, mechanical, photo, and/or thermal degradation. For example, hydrolytic degradation is the process by which moisture penetrates a disposable material and hydrolyzes, for example, ester bonds, thereby breaking down polymers in the material. Without being bound by theory, hydrolytic degradation is thought to proceed through a series of somewhat overlapping steps including: (1) diffusion of water into the material; (2) initial hydrolysis yielding polymers with reduced molecular weight (i.e., conversion of polymers to oligomers); (3) continued loss of molecular weight (i.e., formation of smaller oligomers) and gradual loss of plasticizers incorporated into the material; (4) initial loss of physical properties (e.g., pliability); (5) loss of further properties resulting in an opaque and hazy material; (6) major loss of physical properties, such as tensile strength and form-stability; (7) weight loss; and (8) volume loss, until the material is essentially degraded to monomers or small oligomers. Typically, the obvious loss of physical properties correlates with a reduction in molecular weight of the polymer down to a number average molecular weight of about 50,000 daltons.

For example, one such biodegradable material is poly(hydroxyacids) (“PHA's”) such as polylactic acid (“PLA”) and polyglycolic acid (“PGA”). PHA's have been known for many years. Among the important properties of these polymers are their tendency to depolymerize relatively easily and their ability to form environmentally benign byproducts when degraded or depolymerized. Consequently, high molecular weight PHA polymer shaped articles are finding increasing application as replacements for non-degradable polymers such as polystyrene in throw-away products like fast-food containers (Sinclair et al, WO90/01521, published Feb. 22, 1990).

Additionally, these biodegradable materials are being used as packaging material. U.S. Pat. No. 6,573,340 discloses a biodegradable polymer blend suitable for laminate coatings, wraps and other packaging materials. U.S. Pat. No. 5,883,199 discloses a biodegradable blend that is used to manufacture sheets or films, bags, containers, such as bottles and disposable cups, disposable diapers, and other items

Despite the advances taught by the above-identified patents and applications, the contents of which are incorporated herein by reference, there exists a need for an improved biodegradable packaging material.

### SUMMARY

The present invention provides a biodegradable packaging material useable for indicating the expiration of the shelf-life of the goods enclosed therein. For example, the packaging material of the present invention is used to enclose perishable foods and beverages, medicines, medical devices, medical instruments, medical implant, or other such limited shelf-life goods.

Additionally, the packaging material is used to show when the integrity of the packaging has been compromised. For example, perishable foods or sterilized medical devices are stored in airtight or vacuum sealed packaging. In some instances, the seal is broken without the knowledge of the user. The packaging material of the present invention is used to indicate a broken seal and contamination or spoilage of the goods enclosed therein.

Alternatively, the packaging material is used to intentionally break the seal, contaminating the goods enclosed therein. In some instances medical instruments, devices, or implants have a limited shelf life, the packaging material can indicate the expiration of the shelf life and render the enclosed goods unusable by intentionally contaminating and/or providing a visual indicator of expiration of shelf-life.

In one embodiment, the packaging material for enclosing a packaged good comprises a first film layer, a second biodegradable film layer, and a reactive chemical interposed between the first and second film layers. The second film layer degrades when exposed to a first reactive stimuli, which can be, for example, air or moisture. The reactive chemical can change color when exposed to a second reactive stimuli. The second reactive stimuli can be the same or different than the first reactive stimuli.

In an embodiment, the packaged good is a food product and the second film layer is proximal to the food product. The first reactive stimuli can be an enzyme, bacteria, or chemical emitted from the food product. The reactive chemical can indicate spoilage of the food product.

In another embodiment, the packaged good is a medical device and the second film layer is proximal to the medical device. The reactive chemical can indicate contamination of the medical device. The first film layer can be made of a biodegradable material such that the first film layer degrades when exposed to a third reactive stimuli consisting of air or moisture. If the enclosed product has a limited shelf life and the first film layer degrades, the reactive chemical is exposed to indicate the expiration of the limited shelf life.

The present invention also includes a packaging system for indicating a status of an enclosed good. The system comprises a packaging material enclosing the good and a status indicator incorporated into a least a portion of the packaging material. The status indicator includes first and second film layers, at least one of the first and second film layers being biodegradable, and a reactive chemical interposed between the first and second film layers. At least one of the first and second film layers can degrade when exposed to a first reactive stimuli. The reactive chemical can change color when exposed to a second reactive stimuli.

In another embodiment, a packaging material encloses a perishable product subject to spoiling. The packaging material comprises an inner layer made of a first biodegradable material, an outer layer made of a second biodegradable material, and a reactive chemical interposed between the inner and outer layers. The second biodegradable material is different from the first biodegradable material and degrades at a slower rate than the first biodegradable material. The perishable product releases a stimulus during spoiling and the reactive chemical provides a visual indication of exposure to the stimulus.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts a cross sectional view of an embodiment of the packaging material of the present invention;

FIG. 2 depicts a cross sectional view of an another embodiment of the packaging material of the present invention;

FIG. 3 depicts the packaging material of the present invention enclosing a perishable food;

FIG. 4 depicts a cross sectional view of an additional embodiment of the packaging material of the present invention;

FIG. 5 depicts a cross sectional view of a further embodiment of the packaging material of the present invention;

FIG. 6 depicts a perspective view of an embodiment of the present invention including a freshness or shelf-life indicator;

FIG. 7 depicts a cross sectional view of the freshness or shelf-life indicator of the present invention;

FIG. 8 depicts a perspective view of an embodiment including a contamination button;

FIG. 9 depicts a cross sectional view of a contamination button of the present invention;

FIG. 10 depicts a perspective view of an embodiment including a contamination button and freshness or shelf-life indicator of the present invention;

FIG. 11 depicts a cross sectional view of the contamination button and freshness or shelf-life indicator of the present invention;

FIG. 12 depicts a perspective view of an embodiment of the present invention including fiber members;

FIG. 13 depicts a cross sectional view of the packaging material of the present invention including a variable thickness outer layer;

FIG. 14 depicts a perspective view of an embodiment of the present invention including a variable thickness outer layer and duration markers;

FIG. 15 depicts a cross sectional view of the packaging material of the present invention including a degradation agent dispersed therein;

FIG. 16 depicts a top perspective view of a surgical dressing of the present invention; and

FIG. 17 depicts a bottom perspective view of the surgical dressing of the present invention.

### DETAILED DESCRIPTION

The present invention provides a biodegradable packaging material useable for indicating the expiration of the shelf-life of the goods enclosed therein. For example, the packaging material of the present invention is used to enclose perishable foods or beverages, medicines, medical devices, medical instruments, medical implants, or other such limited shelf-life goods.

Additionally, the packaging material is used to show when the integrity of the packaging has been compromised. For example, perishable foods or sterilized medical devices are stored in airtight or vacuum sealed packaging. In some instances, the seal is broken without the knowledge of the user. The packaging material of the present invention is used to indicate a broken seal and contamination or spoilage of the goods enclosed therein.

Alternatively, the packaging material is used to intentionally break the seal, contaminating the goods enclosed therein. In some instances medical instruments, devices, or implants have a limited shelf life, the packaging material can indicate the expiration of the shelf life and render the enclosed goods unusable by intentionally contaminating and/or providing a visual indicator of expiration of shelf-life.

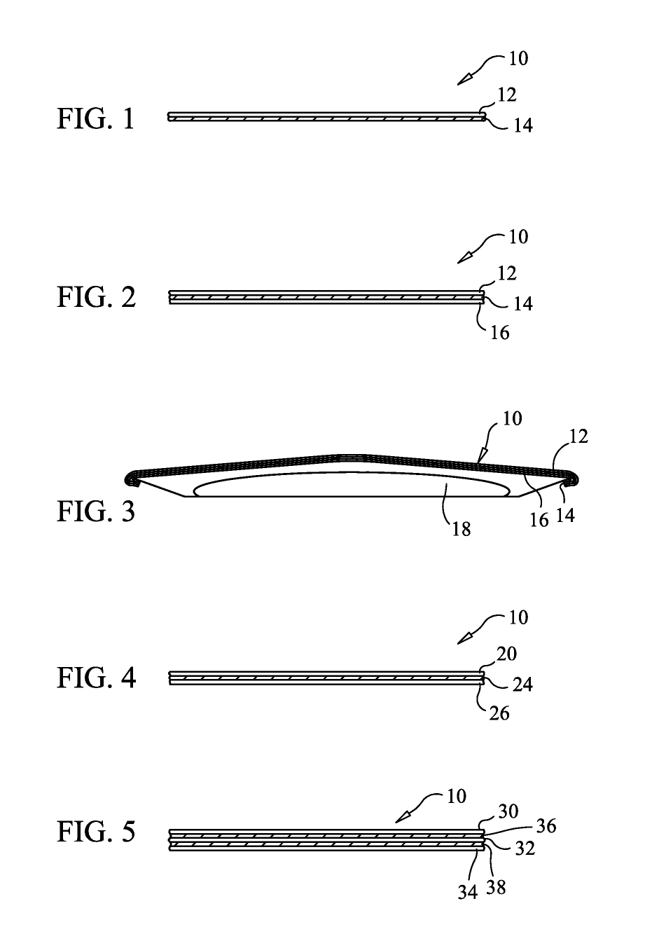

Referring now to the drawing figures in which like reference designators refer to like elements, there is shown in FIG. 1 an embodiment of the packaging material 10 of the present invention. A first film layer 12 is coated with a reactive chemical (chemical indicator) 14, wherein the reactive chemical 14 changes color when exposed to a predefined stimulus. For example, the reactive chemical 14 can change color when exposed to air, oxygen, carbon dioxide, nitrogen, moisture, or other stimuli. When the packaging material 10 is used to vacuum seal a product, the reactive chemical 14 will immediately change color, indicating a break in the vacuum seal.

In one embodiment, the first film layer 12 is made of a biodegradable material and is positioned with respect to the product such that the reactive chemical 14 is adjacent to the product. As the first film layer 12 degrades, the reactive chemical 14 provides an indication that the packaging material 10 has been compromised. Alternatively, the reactive chemical 14 can provide an indication that the packaged product is no longer safe for consumption or use. In another embodiment, the first film layer 12 is adjacent to the packaged product and the reactive chemical 14 is on an exterior surface, i.e. exposed to the environment. In this embodiment, the reactive chemical 14 would be selected to not respond to normal environmental factors. Rather, the reactive chemical 14 could be selected to activate if the ambient environment deviates from a range, e.g. too high or too low of a temperature.

An exemplary reactive chemical 14 which changes color when exposed to moisture can include copper sulfate, which is substantially colorless in a dry, (anhydrous) form, and deep blue when hydrated in its crystal structure, or in solution. Alternatively, cobalt chloride, which is blue in an anhydrous water free state and red in a hydrated state, can be used. Furthermore, there are a number of known suitable chemicals which undergo visible color changes due to a chemical reaction when exposed to atmospheric moisture or other predefined stimulus.

Additionally, the reactive chemical 14 can be a composition which can include anhydrous mixtures of a solid acidic or basic substance, intimately mixed with an organic acid-base indicator dye. This mixture can be strongly diluted by a suitable neutral, solid material, or is disposed on a solid support. In this example, the acid-base indicator undergoes a color change only when moisture has been absorbed from the atmosphere to dissolve the acidic or basic substance and thereby “expose” the indicator dye to the acid or base.

In one embodiment, reactive chemical 14 compositions can be anhydrous, powdered sodium carbonate (basic substance), an acid-base indicator dye such as litmus, phenolphtalein or methyl-orange (sodium p-dimethylamino azo benzene sulfonate) and a suitable support such as a cotton pad or a neutral solid powder. Methyl orange is yellow in a basic medium and orange in acidic medium, phenolphtalein is colorless in neutral or acidic medium, and red in basic medium.

Still other reactive chemical 14 compositions include inorganic and organic dyes. For example leuko-dyes which undergo oxidation to form a visibly colored dye may be used. One example of an organic chemical is an alkaline solution of pyrogallol which is on a suitable solid support. Alkaline pyrogallol is colorless in the absence of oxygen, but turns virtually black when exposed to oxygen. There are a number of other known suitable chemicals which undergo visible color changes due to a chemical reaction when exposed to atmospheric oxygen.

Referring to FIG. 2, a second film layer 16 may be positioned over the reactive chemical 14, sandwiching the reactive chemical 14 between the first and second film layers 12, 16. The second film layer 16 can be a biodegradable film, such that after a specified time period the second film layer 16 will sufficiently degrade, exposing the reactive chemical 14 to reactive stimuli. Biodegradable films are known in the art, for example, Cargill Dow LLC manufactures polylactide pellets from corn starch. The polylactide pellets is made into a biodegradable film by Mitusbishi Plastics, being sold under the name of ECOLUJU.

The degradation rate of the biodegradable film is controlled, such that exposure of the reactive chemical to the reactive stimuli will coincide with the expiration of the enclosed goods. For example, U.S. Pat. No. 6,323,307 discloses a method for controlling the degradation rate of the biodegradable material. It is also known that the degradation rate of PLA/PGA mixtures can be controlled by varying the relative amounts of PLA and PGA.

As previously set forth, the reactive chemical 14 can also be configured to appear over time due to exposure to the environment. In particular, the reactive chemical 14 is responsive to time intervals, temperature levels, oxygen levels, or the like, and combinations thereof. Various visual indicators that appear over time in response to particular conditions are disclosed in U.S. Pat. No. 5,058,088 to Haas et al.; U.S. Pat. No. 5,053,339 to Patel; U.S. Pat. No. 5,045,283 to Patel; U.S. Pat. No. 4,987,849 to Sherman; U.S. Pat. No. 4,903,254 to Haas; U.S. Pat. No. 4,812,053 to Bhattacharjee; and U.S. Pat. No. 4,292,916 to Bradley et al.

Referring to FIG. 3, there is shown the packaging material 10 of the present invention used to enclose a perishable food 18. The moisture of the enclosed food 18 will react with the second film layer 16, causing the second film layer 16 to degrade. As the second film layer 16 degrades, the reactive chemical 14 becomes exposed to the moisture, resulting in a color change. The color change becomes more pronounced with increased or prolonged exposure to the moisture. The degree of color change is used as an indicator of spoilage of the food, wherein a darker or more pronounced color indicates that the food is no longer fit for consumption.

Alternatively, in perishable foods spoilage bacteria cause food to deteriorate more quickly because of their short reproduction times. The spoilage bacteria multiply very rapidly by a process called cell replication or binary fission—one cell divides and becomes two. If conditions such as moisture and temperature are right, for example, certain bacteria can reproduce in as little as 20 minutes. Within 20 minutes, one cell becomes two; in 40 minutes, there would be four, and so on.

As the spoilage bacteria grow, the amount of enzymes produced by those bacteria increases. Enzymes are a normal component of food that help speed up or slow down chemical reactions. The enzymes in a banana, for example, cause it to change color from green to yellow, and then brown to black, as it matures. The ripening and softening of other fruits, such as peaches, tomatoes and apples, are other examples of enzyme action. The numbers of microorganisms or enzymes present on a food product determine the degree of food spoilage.

As discussed above, the moisture present in the packaged foods 18 reacts with the second film layer 16, initiating degradation of the second film layer 16. As the second film layer degrades, the reactive chemical 14 becomes exposed to the enzymes. The reactive chemical 14 can be any chemical species which undergoes a detectable change as a result of the reaction or as a result of the culmination of reactions occurring when the released enzyme is present. The resulting detectable change is an indication that the enzymatically active hydrolase is present. The reactive chemical 14 can be a visual indicator and, for example, a chromogenic indicator, i.e., those in which the visible change is a change in color, including the formation of color in an otherwise colorless material, upon action of the enzyme when it is released from the packaged food 18. With the increased exposure to the enzymes, the color change becomes more pronounced indicating spoilage of the food. The most appropriate chromogenic indicator for any given enzyme will depend upon the reaction or reactions which the enzyme is capable of catalyzing or initiating, and the selection in any given case will be readily apparent to those skilled in the art.

Alternatively, the enzyme may be capable of catalyzing the formation of a fluorescent signal, a phosphorescent signal, a bioluminescent signal, a chemiluminescent signal, or an electrochemical signal upon its release from the packaged food 18. Additionally, the enzyme may be capable of producing other visible or detectable signals, such as, for example, a clot, agglutination, a precipitation, or a clearing zone. In these cases, the reactive chemical 14 would be the chemical species or substrate required by the enzyme in order to bring about the desired detectable change.

As well as reacting with the reactive chemical 14, the enzymes can additionally react with the second film layer 16. The enzymes can increase the rate of degradation of the second film layer 16, which results in an increase in exposure of the reactive chemical 14 to the enzymes.

Additionally, food may deteriorate as a result of chemical changes within the food itself or, more broadly, from temperature abuse. The odor associated with bad food is caused by a chemical reaction that breaks down the molecular chains that make up fatty acids in fat to compounds called aldehydes, and may continue to smaller-sized fatty acids, resulting in the release of offensive or musty odors. This offensive or musty odor is caused by a vaporized or gaseous from of these chemicals. For example, in a perishable food such as fish, the odor is caused by vaporized or gaseous chemicals known as “violatiles.”

As discussed above, the moisture present in the packaged foods 18 reacts with the second film layer 16, initiating degradation of the second film layer 16. As the second film layer 16 degrades, the reactive chemicals 14 becomes exposed to the vaporized or gaseous chemicals. The vaporized or gaseous chemicals react with the reactive chemical 14, resulting in a color change. With the increased exposure to the vaporized or gaseous chemicals, the color change becomes more pronounced indicating spoilage of the food.

In the above examples the second film layer 16 layer is exposed to the enclosed food 18. Alternatively, the second film layer 16 is on the external side of the packaging material 10, being exposed to the air. The moisture in the air causes the second film layer 16 to degrade, exposing the reactive chemical 14 to the air. The air reacts with the reactive chemical 14, resulting in color change. By air it is meant that the reactive chemical 14 reacts with nitrogen, oxygen, carbon dioxide, or any other gas or combinations of gases present in air. With the increased exposure to the air, the color change becomes more pronounced indicating expiration of the shelf-life. This above embodiment is especially useful for long shelf-life food products, such as freeze dried or dehydrated foods.

It is envisioned the either one or both the first and second film layers 12, 16 are transparent or at least translucent. Optionally, the first and/or second film layer 12 and 16 is tinted, including a color, opaque, or include a reflective coating. For example, if the outer film (either first or second film layer 12 and 16) is yellow and the reactive chemical 14 is blue, a green color will show. If reactive chemical 14 turns red upon exposure to a stimulus, an orange color shows.

Referring to FIG. 4, the packaging material 10 of the present invention includes first and second film layers 20, 26, wherein each of the first and second film layers 20, 26 are made of a biodegradable film. Each of the first and second film layers 20, 26 have different degradation rates. A reactive chemical 24 is interposed between the first and second film layers 20, 26, wherein the reactive chemical 24 changes color when exposed to a predefined stimulus.

In such instances, the packaging material 10 is used as an indicator of expiration of shelf-life and/or contamination or spoilage of the enclosed goods. For example, the first film layer 20 is an inner layer and is adjacent to the packaged goods, for example a perishable food. The moisture of the enclosed food will react with the first film layer, causing the first film layer 20 to degrade. As the first film layer 20 degrades, the reactive chemical 24 becomes exposed, reacting with the moisture, enzymes, chemicals produced by the food, or the degradation product(s) of first film layer 20 resulting in a color change. The color change becomes more pronounced with increased or prolonged exposure. The degree of color change is used as an indicator of spoilage of the food, wherein a darker or more pronounced color indicates that the food is no longer fit for consumption.

The second film layer 26 is an outer layer and is used to indicate the expiration of a specified time period, i.e. shelf-life. As noted above, the second film layer 26 is exposed to the air. The moisture in the air causes the second film layer 26 to degrade, exposing the reactive chemical to the air. The air reacts with the reactive chemical, resulting in color change. With the increased exposure to the air, the color change becomes more pronounced indicating expiration of the shelf-life.

Additionally, the packaging material 10 is used to store sterilized products such as medical goods. The packaging material 10 is used to indicate an expiration of shelf-life of the medical goods or a contamination, i.e. a break in the packaging material 10, of the medical goods. As noted above, the packaging material 10 of the present invention includes first and second film layers 20, 26, wherein each of the first and second film layers 20, 26 are made of a biodegradable film. Each of the first and second film layers 20, 26 have different degradation rates. A reactive chemical 24 is interposed between the first and second film layers 20, 26, wherein the reactive chemical 24 changes color when exposed to a predefined stimulus.

The medical goods are sealed within the packaging material 10 in a sterilized condition, where the packaging material 10 is also sterilized. Alternatively, the medical goods are sealed within the packaging material 10 in an un-sterilized condition. The packaging material 10 and packaged goods are then sterilized through, for example, gamma radiation, ethylene oxide, or other known sterilization methods.

To protect the integrity of the sterilized medical goods, the medical goods are vacuumed sealed within the packaging material 10. Alternatively, the medical goods are sealed within the packaging material 10 under pressure, in a dry inert gas, for example nitrogen.

Similarly, the packaging material 10 itself is in a sterilized condition. The first film layer 20, the second film layer 26, or both the first and second film layers 20, 26 are sterilized. The packaging material 10 is sterilized by gamma radiation, ethylene oxide, or other known sterilization methods.

The first film layer 20 is an inner layer and is adjacent to the medical goods, for example a medical implant. If there is a break in the packaging material 10, resulting in a contamination of the medical goods, moisture and air will leak into the packaging material 10. The moisture in the air will react with the first film layer 20, causing the first film layer 20 to degrade. As the first film layer 20 degrades, the reactive chemical 24 becomes exposed, reacting with the moisture resulting in a color change. The color change becomes more pronounced with increased or prolonged exposure. The color change is used as an indicator of contamination of the enclosed goods.

Alternatively, as the first film layer 20 degrades, the reactive chemical 24 becomes exposed, reacting with the air resulting in a color change. The color change becomes more pronounced with increased or prolonged exposure. The color change is used as an indicator of contamination of the enclosed goods.

The second film layer 26 is an outer layer and is used to indicate the expiration of a specified time period, i.e. shelf-life. As noted above, the second film layer 26 is exposed to the air. The moisture in the air causes the second film layer 26 to degrade, exposing the reactive chemical to the air. The air reacts with the reactive chemical, resulting in color change. With the increased exposure to the air, the color change becomes more pronounced indicating expiration of the shelf-life.

The packaging material 10 of the present invention can include at least three film layers, with a reactive chemical interposed between each layer. Referring to FIG. 5, the packaging material 10 of the present invention includes first, second and third film layers 30, 32, 34, wherein reactive chemicals 36, 38 are interposed between each of the film layers 30, 32, 34. At least the first and third film layers 30 and 34 are made of biodegradable films, each having different degradation rates. Alternatively, the packaging material 10 can include multiple film layers each having a reactive chemical interposed there between.

In an exemplary embodiment, the first film layer 30 is on the external side of the packaging material 10, being exposed to the air. The moisture in the air causes the first film layer 30 to degrade, exposing the reactive chemical 36 to the air. The air reacts with the reactive chemical 36, resulting in color change. With the increased exposure to the air, the color change becomes more pronounced indicating expiration of the shelf-life. The first film layer 30 is used as an indicator of expiration of shelf-life.

The third film layer 34 is an inner layer, adjacent to the packaged goods, for example a perishable food. The moisture of the enclosed food will react with the third film layer 34, causing the third film layer 34 to degrade. As the third film layer 34 degrades, the reactive chemical 38 becomes exposed, reacting with the moisture, enzymes, or chemicals produced by the food resulting in a color change. The color change becomes more pronounced with increased or prolonged exposure. The degree of color change is used as an indicator of spoilage of the food, wherein a darker or more pronounced color indicates that the food is no longer fit for consumption. The third film layer 34 is used and an indicator of spoilage of the packaged goods.

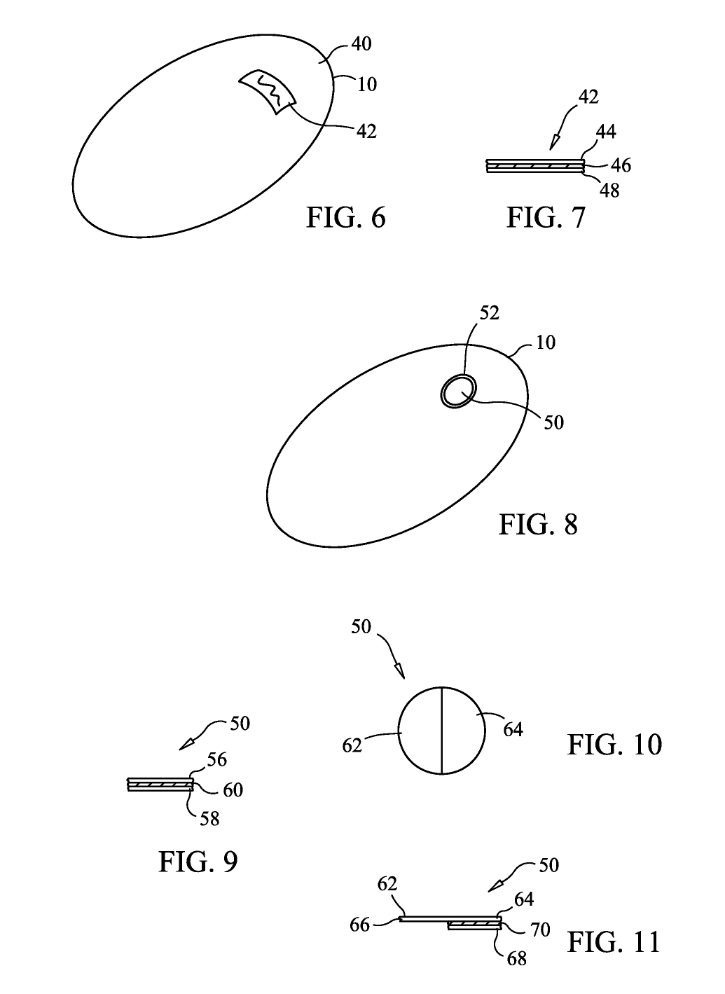

In the above example, the packaging material 10 of the present invention completely or substantially covers the enclosed goods. Referring to FIGS. 6 and 7, the present invention can take the form of an indicator 42 integrated into the packaging material 40. The indicator 42 includes first and second film layers 44, 48, wherein at least one of the film layers 44, 48 is a biodegradable film. A reactive chemical 46 is interposed between the first and second film layers 44, 48, wherein the reactive chemical 46 changes color when exposed to a stimuli. The indicator is incorporated into the packaging, such that the first film layer 44 is an inner layer being exposed to the enclosed goods and the second film layer 48 is an outer film layer being exposed to the air.

As discussed above, in the instance when the first film layer 44 is a biodegradable film, the moisture of the enclosed food will react with the first film layer 44, causing the first film layer 44 to degrade. As the first film layer 44 degrades, the reactive chemical 46 becomes exposed, reacting with the moisture, enzymes, or chemicals produced by the food resulting in a color change. The color change becomes more pronounced with increased or prolonged exposure. The degree of color change is used as an indicator of spoilage of the food, wherein a darker or more pronounced color indicates that the food is no longer fit for consumption.

Alternatively, the second film layer 48 is a biodegradable film layer, being exposed to the air. The moisture in the air causes the second film layer 48 to degrade, exposing the reactive chemical 46 to the air. The air reacts with the reactive chemical, resulting in color change. The color change indicates an expiration of the shelf-life.

Similarly, both the first and second film layers 44, 48 are biodegradable films, wherein the first and second film layers 44, 48 degrade at different rates. In such instance, the indicator is used as an indicator of expiration of self-life and/or contamination or spoilage of the enclosed goods. For example, the first film layer 44 is an inner layer, being adjacent to the packaged goods, for example a perishable food. The moisture of the enclosed food reacts with the first film layer 44, causing the first film layer 44 to degrade. As the first film layer 44 degrades, the reactive chemical 46 becomes exposed, reacting with the moisture, enzymes, or chemicals produced by the food resulting in a color change. The color change becomes more pronounced with increased or prolonged exposure. The degree of color change is used as an indicator of spoilage of the food, wherein a darker or more pronounced color indicates that the food is no longer fit for consumption.

The second film layer 48 is an outer film layer and is used to indicate the expiration of a specified time period, i.e. shelf-life. As noted above, the second film layer 48 is exposed to the air. The moisture in the air causes the second film layer 48 to degrade, exposing the reactive chemical 46 to the air. The air reacts with the reactive chemical, resulting in a color change. The color change indicates an expiration of the shelf-life.

In the instance when the enclosed goods are vacuumed sealed, having little or no moisture present, the packaging material 10 is designed to intentionally break the packaging seal, contaminating the enclosed goods. For example, medical devices are sterilized and vacuumed sealed in the packaging material, preserving the integrity of the sterilized device. In the instance where these devices have a limited shelf-life, the packaging material intentionally contaminates the sterilized device.

Referring to FIG. 8, the packaging material 10 of the present invention includes a “button” 50 made of a biodegradable film, wherein the button 50 is incorporated into the packaging material 10. The button 50 acts as a plug to seal a port 52 in the packaging material 10. The button 50 is exposed to the air, wherein the moisture in the air causes the biodegradable film to degrade. When the button 50 has sufficiently degraded the packaging seal is broken, contaminating the enclosed device.

To indicate the breaking of the packaging seal, the packaging material can include a plurality of dimples. When the packaged goods are sealed within the packaging material in a vacuum the dimples are drawn into the packaging material. When the button 50 has sufficiently degraded, breaking the packaging seal, the dimples pop up indicating the loss of vacuum and contaminating the enclosed device.

Referring to FIG. 9, the button 50 includes a first and second film layer 56, 58, wherein the first film layer 56 is a biodegradable film. A reactive chemical 60 is interposed between the first and second film layers 56, 58. The reactive chemical 60 changes color when exposed to a predefined stimulus. For example, the reactive chemical 60 can change color when exposed to air, oxygen, carbon dioxide, nitrogen, moisture, or other stimuli. The button 50 is integrated into the packaging material 10 such that the first film layer 56 is exposed to the air. The moisture in the air causes the first film layer 56 to degrade, exposing the reactive chemical to the air. The air reacts with the reactive chemical, resulting in color change. The color change indicates the expiration of the shelf-life. Alternatively, the moisture in the air reacts with the reactive chemical 60, resulting in color change. The color change indicates the expiration of the shelf-life.

In another embodiment, the button 50 is integrated into the packaging material 10 such that the first film layer 56 is exposed to the interior of the packaging. The moisture of the enclosed food will react with the first film layer 56, causing the first film layer 56 to degrade. As the first film layer 56 degrades, the reactive chemical 68 becomes exposed, reacting with the moisture, enzymes, or chemicals produced by the food resulting in a color change. The color change becomes more pronounced with increased or prolonged exposure. The degree of color change is used as an indicator of spoilage of the food, wherein a darker or more pronounced color indicates that the food is no longer fit for consumption.

Alternatively, in the instance where the goods are vacuumed sealed, a break in the seal will expose the interior of the packaging to air and moisture. The moisture in the air causes the first film layer 56 to degrade, exposing the reactive chemical 60 to the air. The air reacts with the reactive chemical, resulting in color change. The color change indicates a break in the vacuum seal. Alternatively, the moisture in the air reacts with the reactive chemical 60, resulting in color change.

Similarly, it is contemplated that the button 50 can include a single film layer 56 coated with a reactive chemical 60. The button 50 is position on the packaging such that the reactive chemical is exposed to the interior of the packaging. In the instance where the goods are vacuumed sealed, a break in the seal will expose the interior of the packaging to air and moisture. The air immediately reacts with the reactive chemical 60, resulting in color change. The color change indicates a break in the vacuum seal. Alternatively, the moisture in the air reacts with the reactive chemical 60, resulting in color change.

Referring to FIGS. 10 and 11, the button 50 can include two sections, a first section 62 which acts as a plug to seal the port 52 in the packaging material 10 and a second section 64 which indicates expiration of shelf life. The first section 62 is made of a first biodegradable film layer 66. The first biodegradable film layer 66 is exposed to the air, wherein the moisture in the air causes the first biodegradable film layer 66 to degrade. When the first biodegradable film layer 66 has sufficiently degraded the packaging seal is broken, contaminating the enclosed device.

The second section 64 includes the first biodegradable layer 66 and a second film layer 68, wherein a reactive chemical 70 is interposed between the first and second film layers 66. The reactive chemical 70 changes color when exposed to a predefined stimulus. For example, the reactive chemical 70 can change color when exposed to air, carbon dioxide, nitrogen, moisture, or other stimuli. The moisture in the air causes the first film layer 66 to degrade, exposing the reactive chemical 70 to the air. The air reacts with the reactive chemical 70, resulting in a color change. The color change indicates the expiration of the shelf-life. Alternatively, the moisture in the air reacts with the reactive chemical 70, resulting in color change. The color change indicates the expiration of the shelf-life.

The above packaging material 10 is inherently disclosed as having a single chamber for enclosing the goods. However, it is contemplated, that the packaging material 10 includes multiple chambers, each individually containing a single, or a portion of the goods. The multi-chamber packaging material allows a selected portion of the goods to be used, without contaminating the remaining portions of the goods.

The packaging material 10 of the present invention is pliable for forming about the packaged goods. The packaging material 10 is shrink wrapped, vacuumed sealed, or combination thereof about the packaged goods. For example, the packaging material 10 formed over, about, the packaged good with application of low temperature heat, such as applied by a hot air gun and the like. Additionally, the packaging material 10 is sealed using heat, wherein the packaging material 10 heat seals at low temperatures.

Alternatively, the packaging material 10 is substantially rigid material which becomes malleable with the application of low temperature heat, such as applied by a hot air gun and the like. The shape of the packaging material 10 can be altered to accommodate the enclosed goods for storage. For example, the packaging material 10 may have an initial cylindrical shape. With the application of heat, the packaging material can be transformed into a rectangular shape to allow for ease of storage.

The packaging material 10 is sealed using a mechanical fastener, for example a “Ziplock-type” ® fastener (Ziplock® is a trademark owned by SC Johnson), hook and loop fasteners, or other similar type mechanical fasteners. The hook and loop fasteners can be biodegradable, as described in co-pending U.S. patent application Ser. No. 10/427,151, entitled “Tissue Fastener and Method of Using Same.”

The packaging material 10 can be further sealed using heat. The mechanical fastener provides an initial seal to the packaging material 10. Thereafter, heat is applied to the mechanical fastener, bonding the opposing ends of the packaging material 10 together, sealing the packaging material 10.

Alternatively, the packaging material 10 is sealed under pressure, wherein the internal pressure is greater than the external pressure, atmospheric pressure. The packaged goods are in a pressurized gas which includes, but not limited to, air, oxygen, argon, nitrogen, or other inert gas. For example, the packaging material 10 can be sealed under high pressure, where the high pressure will increase the shelf life of food product, delaying food spoilage.

The pressure in the sealed packaging material 10 is adjusted using a self sealing port integrated into the packaging material 10. The self-sealing port allows for the pressure in the packaging material 10 to be increased or decreased without contaminating the sealed goods. For example, the pressure is increased by inserting a needle through the self-sealing port and injecting a gas into the packaging material 10. The self-sealing port seals about the needle, preventing gas from leaking out of the packaging material 10 at the injection site. The injected gas is of the same content as is present in the packaging material 10 to prevent contamination of the sealed goods. As the needle is removed, the self-sealing port seals the hole formed by the needle preventing gas from leaking out of the injection site.

To decrease the pressure in the packaging material 10, a needle is inserted through the self-sealing port and gas is removed from the packaging material 10. The self-sealing port seals about the needle, preventing gas from seeping into the packaging material 10 at the injection site. Sufficient gas is removed, such that the pressure in the packaging material 10 is less than external pressure. Alternatively, substantially all of the gas is removed from the packaging material 10, vacuum sealing the enclosed goods. As the needle is removed, the self-sealing port seals the hole formed by the needle, preventing gas from seeping into the packaging material 10 through the injection site.

The packaging material 10 can be substantially rigid forming a hard shell container for receiving the packaged goods. The hard shelled container is vacuumed formed or formed through injection molding. To add rigidity to the container, the container can include stiffening elements. The stiffening element can take the form of textured, ribbed, or waffle sections formed in the container or a variable thickness in the surface of the material. For example, the surface of the packaging material can include longitudinal ridges as stiffening elements. It is envisioned that any three dimensional surface formation is used for stiffening the packaging material.

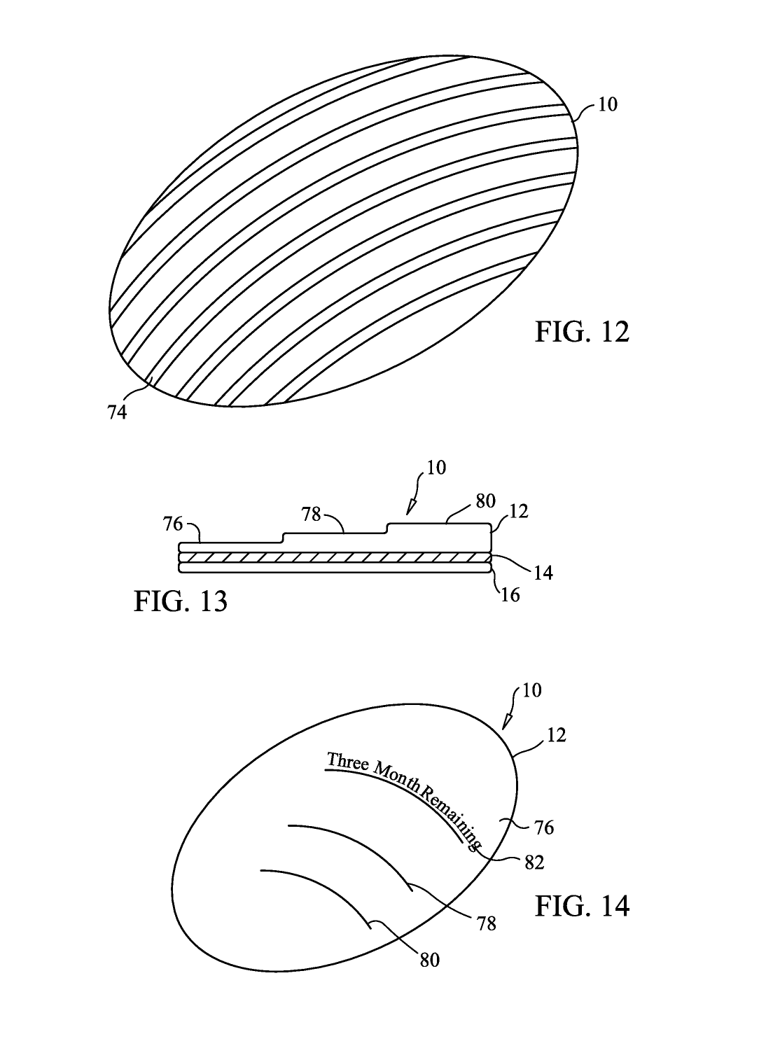

In the above description, the packaging material is made from a sheet of a biodegradable material. Referring to FIG. 12, it is contemplated that the packaging material 10 can include fiber members 74 there through. The fiber members 74 are made of biodegradable or non-biodegradable materials. For example, the fiber can include cotton, cellulose, or other organic product.

The fiber members 74 act as stiffeners, providing rigidity to the packaging material 10. Furthermore, the fiber members 74 have different heat reactive properties than the packaging material 10. For example, the fiber members 74 deform at a lower temperature than the packaging material 10, allowing the fiber members 74 to conform to the packaging material 10 about the packaged goods or to form any custom shape.

Additionally, it is contemplated that the packaging material 10 is formed from a plurality of fibers joined together. The fibers are woven together to form biodegradable sheets. Alternatively, the fibers joined together using bonding agents to form the biodegradable sheets. For example, the fibers are joined together using a biodegradable epoxy.

As noted above, some of the fiber members 74 are made of biodegradable or non-biodegradable materials. Additionally, the sheets include different fiber members having different mechanical properties, wherein some of the fibers act as stiffeners, providing rigidity to the packaging material 10. Furthermore, some of the fiber can have different heat reactive properties, deforming at a lower temperatures, allowing the fibrous sheet to be conformed about the packaged goods or to form any custom shape.

Referring to FIGS. 13 and 14, the degradation rate of the packaging material 10, and exposure of the reactive chemical 14, is dependent on the thickness of the film layers 12, 16. Shorter shelf-life goods will have a thinner outer film layer (first layer 12), allowing the reactive chemical 14 to be exposed in a short time frame. Similarly, longer shelf life goods will have a thicker outer layer 12, increasing the time for exposure of the reactive chemical 14. It is envisioned that outer film layer 12 can have varying thickness, as to provide a time marker for remaining shelf-life. For example, the outer layer 12 can have three levels of thickness 76, 78, and 80, where in the first level 76 degrades, thereby exposing a portion of the reactive chemical 14, three months prior to the end of the shelf life. The reactive chemical 14 will change color, indicating exposure. Markings 82 adjacent to the exposed reactive chemical 14 are used to indicate the remaining shelf-life term, for example, “Three Months Remaining” The remaining two levels 78 and 80, will degrade monthly (for example), until the end of the shelf-life. Each exposure of the reactive chemical 12 is used to indicate remaining shelf-life term.

The degradation rates of the packaging material 10 are controlled using a non-reactive coating. For example, the surface of the packaging material 10 is coated with a gelatin material or other polymeric layers. The gelatin material is chemical neutral, thereby preventing degradation until removed. Alternatively, the gelatin material can include enzymes to increase the rate of degradation.

The degradation rates of the packaging material 10 are further increased by the inclusion of chemically reactive agents into the packaging material 10. The chemically reactive agents is mixed in or bonded with the packaging material 10 upon formation. Alternatively, the rate of degradation is controlled with the inclusion of photosensitive material in the packaging material 10. The photosensitive material will increase the rate of degradation when the packaging material 10 is exposed to radiation of a given wavelength, for example visible light or ultraviolet light.

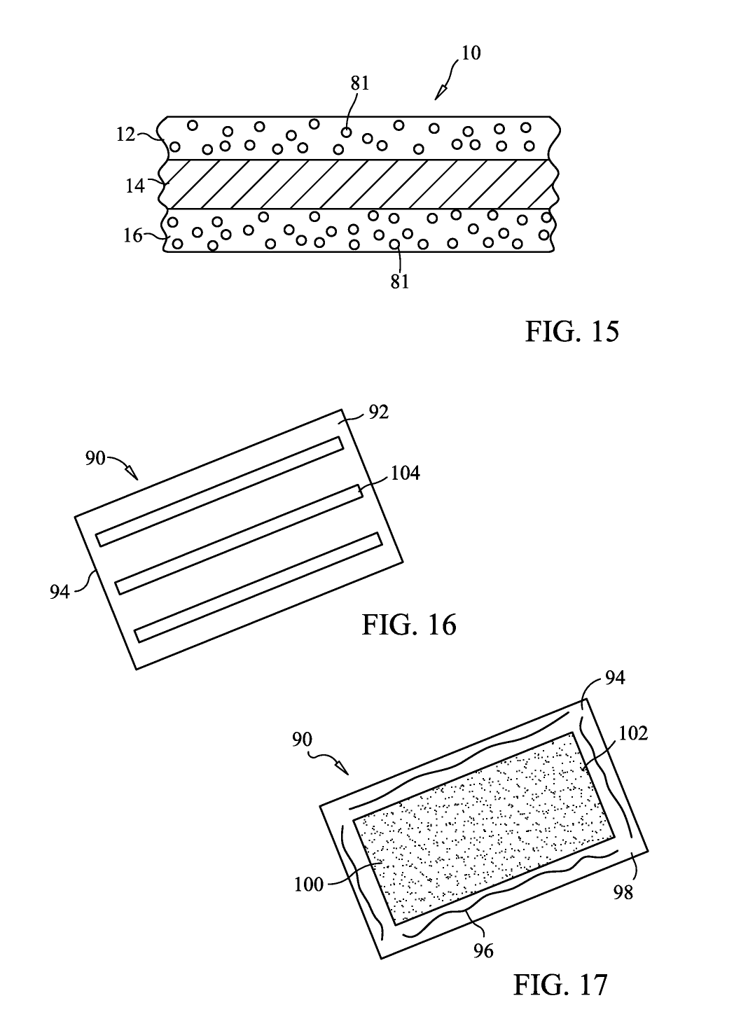

Referring to FIG. 15, to increase the rate of degradation of the packaging material 10 after the packaging material 10 has been discarded, degradation agents 81 are incorporated into the layers 12 and 14 of the packaging material 10. The degradation agent 81 can be fertilizing agent mixed in or bonded with the packaging material 10 upon formation. The fertilizing agents 81 can include sulfates, nitrates, and/or other compounds that make soil more fertile.

Alternatively, degradation agents 81 are active agents. The active agents 81 are maintained in a stasis, being sealed within the layers 12 and 16. As the layers 12 and 14 degrade, the active agents 81 are exposed to air and moisture, activating the active agents 81, increasing the degradation rate of the packaging material 10. The active agent 81 can include salts, acidic agents, molds, or bacteria. For example, the active agents 81 are salts. When exposed to moisture the salts form an acid, increasing the degradation of the packaging material 10.

It is also envisioned that the packaged goods is made of or includes biodegradable materials. For example, medical devices, medical instruments, and medical implants can be made to include a biodegradable material. For example, medical implants are made of a biodegradable material, such that the medical implant will degrade or partial degrade in the body. Such medical implants can include, but not be limited to, tissue fixation devices, stents, sutures, joint repair device, etc.

The medical implants are partially made of a biodegradable material, such that only a portion of the medical implant degrades. In the case where the medical implant is impregnated with an agent, the agent is time released as the biodegradable material degrades. Alternatively, where the medical implant is designed to allow bone ingrowth, such as being made of a porous material, the degradation of the biodegradable material allows for bone ingrowth into the medical implant. Similarly, disposable medical devices and medical instruments, such as, needles, cannulas, IV bags, etc, are made of a biodegradable material. As described above, the biodegradable material can be used to aid the disposal of the device as well as being used to indicate expired shelf life or contamination.

Similarly, the biodegradable material is used to limit the useful life of the medical devices and instruments. For example, in single use devices, such as surgical instruments, scalpels, syringes, and the like, at least a portion of the medical device is made of the biodegradable material. After a single use, the biodegradable portion of medical device degrades, preventing reuse of the device.

The device is packaged in the packaging material 10, in a protective environment to maintain the integrity of the device. As discussed above, if the packaging material 10 is damaged, or the environment contaminated, the packaging material 10 will indicate that the packaged device is no longer fit for use.

As with the medical devices, the goods are packaged in the packaging material 10, providing a protective environment to maintain the integrity of the goods. As discussed above, if the packaging material 10 is damaged, or the environment contaminated, the packaging material 10 will indicate that the package goods are no longer fit for sale to a consumer.

The packaged products can also include other medical related devices which require sterilization such as, surgical gowns, drapes, hoods, protective shield, sponges, bandages, etc. The biodegradable material is used to aid in the disposal of the products as well as being used an indicator of the expiration or contamination of the product.

In the instances wherein the product is made of a fabric, such as clothing, for example, surgical gowns, the biodegradable material is in the form of fibers woven together to form the fabric. It is envisioned that biodegradable, nonbiodegradable, synthetic, and natural fibers, such as cotton, is included in the formation of the fabric. The fabric can also include synthetic fibers made from a fluid repellent material, such as NANO-TEX or TEFLON.

The fabric can include conformal fibers having different heat reactive properties, where some of the fibers become more pliable when exposed to low temperature heat. The low temperature fiber is used to conformal fit the clothing to the wearer. For example, a shirt is partially or wholly made of heat reactive fibers, which react at lower temperature heat, such as body temperature. When the wearer adorns the shirt, the reactive fibers conform the shirt to the wearer's body.

The conformal properties of the conformal fibers include a decrease in fiber length shrinking the garment, such that the clothing will conform to fit tightly about a wearer. For example, a pair of gloves is partially or wholly made of heat reactive fibers. When the wearer adorns the gloves, the fibers shrink, conforming the gloves to the hands of the wearer.

The conformal fibers can be selective woven into the article of clothing, such that only a portion of the article of clothing will shrink. For example, in socks, the top portion of the sock includes the conformal fibers. After the wearer adorns the sock, the conformal fibers shrink to fit the top portion of the sock about the leg of the wearer, preventing the sock from slipping. The conformal fibers is used to replace, or in the alternative supplement, the elastic portion of clothing. It is envisioned that conformal fibers are included in or make any article of clothing, including, but not limited to, pants, shirts, sock, bathing suits, undergarments, such as underwear and brassieres, etc, disposable clothing, such a surgical gowns, and the like.

In products which require an increased absorption, such as a surgical dressing, a wicking agent like fiber (either biodegradable or nonbiodegradable and synthetic or natural fiber), such as cotton, can be included. The absorbing agent can include brushed or roughened surfaces to increase the surface area for absorption. The surgical dressing can further include a therapeutic agent such as an antibiotic incorporated therein. As with surgical dressings, it is envisioned that hygiene products using sheets, woven, and non-woven fibers, such as, diapers and feminine hygiene products are made from biodegradable materials.

Referring to FIGS. 16 and 17, the surgical dressing 90 made of a biodegradable film can be used to cover a treatment site, which can include a wound or incision in a patient. The surgical dressing 90 includes a first surface 92 and a second surface 94, wherein the second surface 94 is positionable on the patient to cover the treatment site. The biodegradable film is sufficiently pliable to conform to the shape of the surface of the treatment site. To conform the surgical dressing to the patient, the surgical dressing 90 is vacuum formed on the patient covering the treatment site. Furthermore, low temperature heat can be applied to the surgical dressing 90 to increase pliability, allowing the surgical dressing 90 to more readily conform to the treatment site.

Alternatively, the biodegradable film has a shape retaining rigidity. With the application of low temperature heat, for example body temperature, the biodegradable film becomes sufficiently pliable to conform to the surface of the treatment site. Heat can also be applied from an external heat source, such as a hot air gun, to increase pliability, allowing the surgical dressing 90 to more readily conform to the treatment site.

The second surface 94 can include an adhesive coating 96 for securing the surgical dressing 90 to the patient. The adhesive coating 96 can cover the peripheral edge 98 of the second surface 94, sealing the surgical dressing 90 over the treatment site. The second surface 94 can additionally include a therapeutic agent, which is dispersed through the treatment site into the patient.

The inner section of the second surface 94 includes an adsorbent pad 100. The absorbent pad 100 includes a plurality of fiber members for absorbing any fluid discharge from the treatment site. The fiber members can include biodegradable fiber members and/or non-biodegradable fiber members. The absorbent pad 100 can further include a non-stick coating or fibers, which prevents the absorbent pad from sticking to the treatment site, i.e. the wound.

In the above description, the surgical dressing 90 is made from a sheet of a biodegradable material. However, it is contemplated that the surgical dressing 90 can include fiber members 102 there through. The fiber members 102 are made of biodegradable and/or non-biodegradable materials. The fiber members 102 act as stiffeners, providing rigidity to the surgical dressing 90. Furthermore, the fiber members 102 have different heat reactive properties than the surgical dressing 90. For example, the fiber members 102 deform at a lower temperature than the surgical dressing 90, allowing the fiber members 102 to conform the surgical dressing 90 about the treatment site or to form any custom shape.

Additionally, it is contemplated that the surgical dressing 90 can be formed from a plurality of fibers joined together. The fibers are woven together to form biodegradable sheets. Alternatively, the fibers are joined together using bonding agents to form the biodegradable sheets. For example, the fibers are joined together using a biodegradable epoxy.

As noted above, some of the fibers are made of biodegradable or non-biodegradable materials. Additionally, the sheets can include different fibers having different properties, wherein some of the fibers act as stiffeners, providing rigidity to the surgical dressing. Furthermore, some of the fibers can have different heat reactive properties, deforming at a lower temperatures, allowing the fibrous sheet to be conformed about the treatment or to form any custom shape.

The surgical dressing 90 of the present invention can include surgical gauze or surgical sponges. The surgical gauze includes a plurality of fiber members for absorbing any fluid discharge for the treatment site. The fiber members can include biodegradable fiber members and non-biodegradable fiber member. The surgical gauze can further include a non-stick coating, which prevents the adhesion to the treatment site, i.e. the wound.

It is also envisioned that the consumer products can include electronic products such as wiring, fiber optic cable, computer chips, batteries, etc. The products is wholly or partially made from or coated with a biodegradable material. Where the electronic components have a limited useful life, the biodegradable material is used to indicate an expiration of that lifetime. This built in obsolescence is a benefit to both the manufacturer (creating a market demand for products) and consumers (indicating time to update technology).

Similarly, the biodegradable material is used to limit the useful life of any goods, devices, or instruments. This can include, but not be limited to, not only medical instruments, but also, electronic devices, electronic readable media, such as DVDs, CDs, memory cards, etc., where at least a portion of these devices are made of the biodegradable material. After an initial exposure to the air, the biodegradable material will begin to degrade, rendering the goods unusable after a set time period.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

What is claimed is:

1. A medical product with a biodegradable portion, comprising:
a medical product with a biodegradable portion, the biodegradable portion made of a biodegradable material and the biodegradable portion degrades faster than the remaining portion of the medical product.

2. A medical product of claim 1, wherein the medical product is at least one of a medicine, medical device, medical instrument, and medical implant.

3. A medical product of claim 1, wherein the medical product is impregnated with an agent.

4. A medical product of claim 3, wherein the agent is time released.

5. A medical product of claim 2, wherein the implant is a material that allows bone growth.

6. A medical product of claim 1, wherein the medical product includes at least one of a needle, cannula, and IV bag.

7. A medical product of claim 1, wherein the medical product includes at least one of surgical instrument, scalpel, and syringe.

8. A medical product of claim 1, wherein the medical product is laminated.

9. A medical product of claim 1, which further includes a stiffening element.

10. A medical product of claim 1, which further includes a fiber member and the fiber member provides rigidity to the packaging material.

11. A medical product of claim 1, wherein the medical product includes a non-biodegradable portion.
